(19) United States
(12) Patent Application Publication
(10) Pub. No.: US 2017/0263017 A1
(43) Pub. Date: Sep. 14, 2017

(54) SYSTEM AND METHOD FOR TRACKING GAZE POSITION

(71) Applicant: Quan Wang, New Haven, CT (US)

(72) Inventor: Quan Wang, Dongguan (CN)

(21) Appl. No.: 15/456,544

(22) Filed: Mar. 12, 2017

Related U.S. Application Data
(60) Provisional application No. 62/306,933, filed on Mar. 11, 2016.

(51) Int. Cl. G06T 7/73; G06T 7/50; G02B 27/01; G06T 7/60; A61B 3/113; G02B 27/00

(52) U.S. Cl. CPC G06T 7/73; A61B 3/113; G02B 27/0093; G02B 27/01; G06T 7/60; G06T 7/50; G06T 2207/30196; H04N 5/33

(57) ABSTRACT

A system for tracking gaze position is provided. In one embodiment, the system includes a first imaging unit, a second imaging unit that can provide depth data. The first imaging unit is configured to acquire subject image data, the second imaging unit is configured to acquire object image data, and the second imaging unit is configured with depth sensor or computation algorithm to acquire object depth data of objects at different depths in a three dimensional environment. A control unit is configured to receive the subject image data, the object image data and the object depth data and calculate a gaze position based on the data. A method for tracking gaze position is also provided.

### BACKGROUND OF THE INVENTION

Control of eye movement is developed in early infancy in human beings. The eyes receive visual sensory information while eye movement is controlled by motor behavior that actively selects where to look. Eye tracking by providing accurate spatial and temporal information of where, what and how the subject is looking has a wide range of applications in vision and behavior researches, as well as for facilitating cognitive studies and psychiatric diagnosis. It also has been applied in computer games, design and marketing investigations, education and training. Eye tracking is a recognized field of study, as various academic conferences such as Eye Tracking and its Researches and Applications (ETRA) have been held to share research, along with publications such as the Journal of Eye Movement Research.

Current eye tracking technology can be classified into two categories, head-mounted eye trackers and screen-integrated eye trackers. They both have pros and cons. The head-mounted eye tracker, a wearable device, has the advantage of its mobility, and is usually used to track eye movements in a real life scene. Its disadvantage is that wearing a helmet or a special pair of glasses with visible cameras may influence the psychological status of subjects since it is not reflective of everyday habits, and wearing the device itself is very difficult with young children or subjects with special needs. Wearing a device is thus a concerning factor in psychological, cognitive or psychiatric measurements. In contrast, the conventional screen integrated eye tracker needs no devices near the subject's head or visual field. However, by utilizing computer screens, the system loses its mobility as all the stimuli or activities are shown on the screen, requiring the subject to remain positioned in front of the computer screen. In addition, screen displays for eye tracking studies have been criticized for a long time because objects and scenes shown on the 2-dimensional planar screen are very different from real life and the 3-dimensional world. Differences include object and scene display sizes and dimensions, and the way that these objects and scenes interact with the subject from the standpoint of psychological effect.

Another issue with conventional technology is the subjective nature of review that takes place for several video coding technologies. Video coding, requires a review of the recorded video of the subject, frame by frame, so that reviewers can use personal judgment as to the most likely place the subject was looking at that frame of time. In psychology labs, behavior video coding is the one of the most tedious, repetitive and time consuming tasks, not to mention the process has subjective inaccuracies of judging the gaze position and inconsistencies between experimenters. 5-10 minutes of video can very easily can take each coder 1 hour to code, and it is intensive work.

What is needed in the art is a system and method for tracking gaze position that allows for greater freedom of movement and less restriction for the subject, while simultaneously providing the subject with a more realistic environment for observation and collection of data.

### SUMMARY OF THE INVENTION

In one embodiment, a system for tracking gaze position of a subject includes a first imaging unit, a second imaging unit operably connected to a control unit; wherein the first imaging unit is configured to acquire subject image data, the second imaging unit is configured to acquire object image data, and to acquire object depth data; and wherein the control unit is configured to receive the subject image data, the object image data and the object depth data and calculate a gaze position based on the received subject image data, object image data and object depth data. In one embodiment, the second image unit has a depth sensor that provides object depth data. In one embodiment, the second image unit is composed of two or more imaging subsystems. In one embodiment, the depth sensor is composed of two or more imaging subsystems. In one embodiment, the first imaging unit is an infrared imaging unit. In one embodiment, the first imaging unit includes an infrared filter. In one embodiment, the first imaging unit samples at a rate of 120 Hz or higher. In one embodiment, at least one of the first and second imaging units include a wireless transmission component for wireless communication with the control unit. In one embodiment, the system includes an infrared light source for generating a corneal reflection that is captured by the second imaging unit.

In one embodiment, a method for tracking gaze position of a subject includes the steps of positioning a first imaging unit and a second imaging unit in an environment including a subject, a first object and a second object, the first and second object at different depths in the environment relative to the subject; determining a first distance between the first imaging unit and the subject based on an image captured from the first imaging unit; determining second distance between a first pupil of the subject and second pupil of the subject based on an image captured from the first imaging unit; determining third distance between the second imaging unit and at least one of the first and second object; and determining a gaze position to one of the first and second objects based on the first, second and third distance. In one embodiment, the first imaging unit and the second imaging unit are positioned back to back. In one embodiment, the first imaging unit and the second imaging unit are spaced apart. In one embodiment, both the first and second imaging units are fixed to a position that is disconnected from the subject. In one embodiment, the method includes positioning an infrared light near at least one of the first and second object. In one embodiment, the method includes illuminating the first and second pupil with infrared light. In one embodiment, the method includes detecting a corneal reflection of the infrared light. In one embodiment, the method includes tracking movement of the subject using the first imaging unit. In one embodiment, the method includes tracking movement of at least one of the first and second object using the second imaging unit. In one embodiment, the first and second imaging units are mounted in a moving vehicle, and wherein the first and second objects are outside of the moving vehicle. In one embodiment, the method includes tracking head movement by utilizing a position marker affixed to the subject. In one embodiment, a distance between the first imaging unit and the subject is determined based on a size of the position marker captured by the first imaging unit. In one embodiment, the method includes tracking head movement by utilizing a face detection and facial features based algorithm.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a diagram of a system for tracking gaze position according to one embodiment.

FIG. 2A is a diagram of a system configuration according to one embodiment, and FIG. 2B is a diagram of a system configuration according to another embodiment.

FIG. 3A is a diagram of an object including an infrared light array according to one embodiment. FIG. 3B is an image of a subject's eye illustrating the pupil area and the corneal reflection, and FIG. 3C is an image of a positional marker on the subject's forehead according to one embodiment.

FIG. 4 is an illustrative diagram of a technique for calculating a subject's gaze position according to one embodiment.

FIG. 5A is a diagram of objects at three different depth levels in a three dimensional space, and FIG. 5B is a diagram illustrating the determination of convergence based on eye position.

FIG. 6 is a flow chart of a method for tracking gaze position according to one embodiment.

FIG. 7 is a flow chart of a method for tracking gaze position according to one embodiment.

FIG. 8 is a diagram of a handheld system for tracking gaze position according to one embodiment.

FIG. 9A is an experimental setup of a gaze tracking system according to one embodiment. FIG. 9B is an image of a left eye and FIG. 9C is an image of a right eye both showing the pupil center and corneal reflection. FIG. 9D is a graph of data depicting distance between pupils.

FIG. 10A is a diagram of a handheld system with imaging subsystems for tracking gaze position according to one embodiment. FIG. 10B is an image of an experiment results of real world scene projection on a 360 degree panorama display and the gaze point of the subject.

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in systems and methods for tracking gaze position. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is a system and method for tracking gaze position.

In one embodiment, a dual imaging unit eye tracking system measures eye movement and gaze positions of a human subject. There are no devices positioned near or connected to the subject's head, and the imaging units can measure the subject's eye movement towards objects in a 3D real-life scene instead of 2D surface of monitor screen. Alternative embodiments do however include a head mounted eye camera, although it is not necessary according to embodiments disclosed herein. According to certain embodiments, the imaging system includes two video cameras, an eye camera and a scene camera, pointing in opposite directions. The eye camera is records images of the subject's head and eye movement, while the scene camera simultaneously records the 3-dimensional real world scene that the subject is observing, including depth information of objects in the scene. By using binocular vergence (e.g. the distance between left and right pupil), the depth that the subject is focused on can be provided to a calibration model. The calibration model is based on the images recorded from the scene camera and based on the real world 3-dimensional scene. In addition to the subject's binocular vergence information, the calibration model also received the object location and depth at the corresponding moment the subject is looking at, based on images recorded from the scene camera and the depth sensor captured of the 3D real world scene. The system accounts for and stabilizes the subject's head movement. Finally, the subject's gaze positions are analyzed, calibrated and visualized. Advantageously, the subject under observation is not distracted or encumbered by a device attached to their body or positioned near their eyes. Further, the subject observes a 3D real-life scene instead of a simulated scene on a 2D screen. Systems and method according to the embodiments described herein facilitate the collection of data superior to data collected by conventional systems, since the subject is allowed to experience an observation environment that is both less restrictive and more realistic.

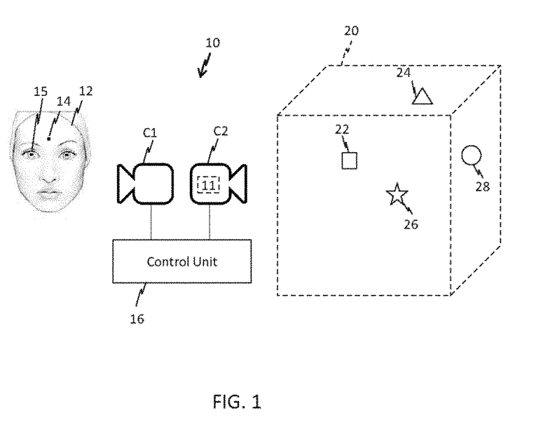

With reference now to FIG. 1, a system 10 according to one embodiment includes two imaging units represented by a first camera C1 and a second camera C2 operably connected to a control unit 16. The first camera C1 is an eye camera trained on the subject 12. The subject's eyes 15 are captured by the first camera C1, along with in certain embodiments a stabilization marker 14 affixed to the center of the subject's forehead. The second camera C2 is the scene camera trained on the 3D scene 20. The 3D scene can be any staged or real-life scene that has one or more objects 22, 24, 26, 28 positioned throughout the scene 20 at two or more different depths. The scene camera C2 also includes a depth sensor 11 for determining the depth of objects in the scene 20. The depth sensor 11 can be an integrated component or function of the scene camera C2, or it can be a separate component from the scene camera C2 that forms an independent connection to the control unit 16.

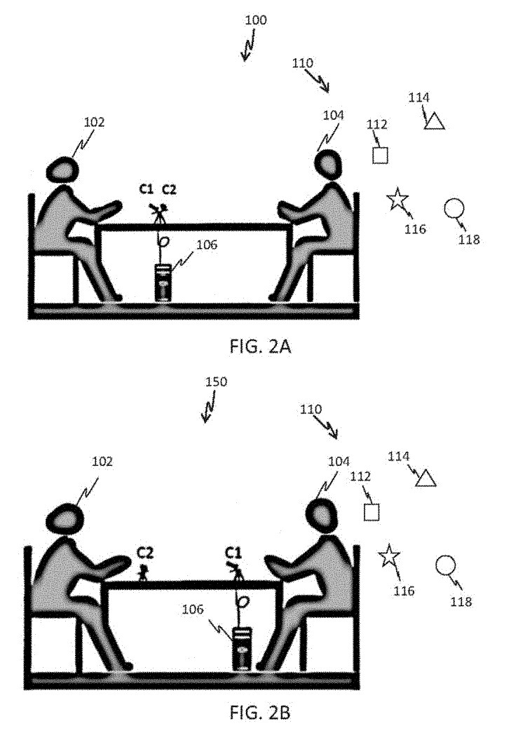

FIGS. 2A and 2B represent embodiments of the system is various setup configurations. In the configuration 100 of FIG. 2A, a subject 102 sits across a table from an interviewer 104. The interviewer 104 is sitting in part of the 3D scene 110 observed by the subject 102, which also includes a number of stationary or moving objects 112, 114, 116, 118. Generally, the 3D scene 110 can be considered anything within the subject's 102 view, which can also include the cameras C1, C2 and the interviewer 104. In certain embodiments, the interviewer 104 is not present in the scene, and instead communicates with the subject 102 from a remote location. On the table positioned between the subject 102 and interviewer 104 is an eye camera C1 directed towards the subject 102 and a scene camera C2 directed towards the 3D scene 110. In this embodiment, the cameras C1, C2 are in a back-to-back configuration on the table. In alternate embodiments, such as the setup 150 illustrated in FIG. 2B, the cameras are separated, with the eye camera C1 positioned deeper into the scene 110, while the scene camera is positioned closer to the subject 102. Both cameras C1, C2 are operably connected to a control unit 106 such as a computer module to send and receive data and instructions. In FIG. 2A, both cameras C1, C2 are connected to the control unit 106 via a hardwired connection. In FIG. 2B, the eye camera C1 is connected to the control unit 106 via a hardwired connection, while the scene camera is connected to the control unit via a wireless connection.

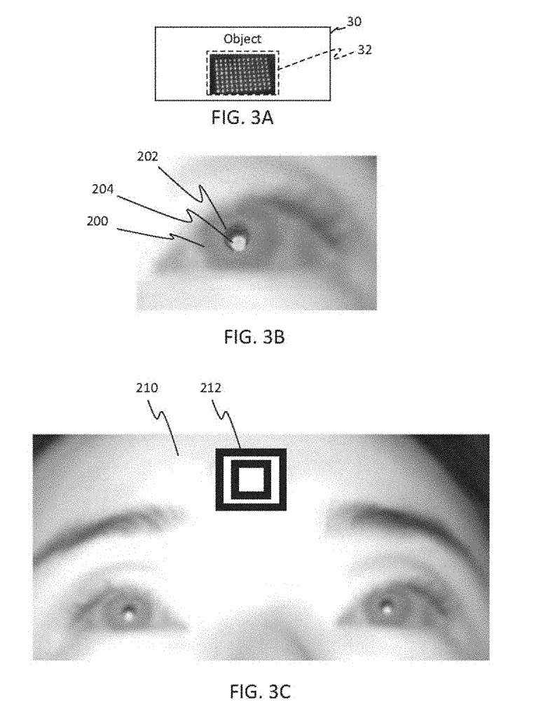

Although cameras are described for use with certain embodiments, any suitable imaging unit can be utilized with the embodiments as would be apparent to those having ordinary skill in the art. The eye camera C1 can be a conventional camera known in the art, such as the type of cameras used in conventional screen-integrated eye tracker systems. In certain embodiments, the eye camera is in infrared camera that can catch the pupil position and the corneal reflection against infrared light. The infrared light source can be integrated with or positioned near one or more objects (e.g. FIG. 3A), or positioned in a general area pointed towards the subject's eyes. In one embodiment, the eye camera is an infrared camera with a capture and focus range adjustable lens, use in combination with an infrared filter. In one embodiment, the eye camera samples at a rate of 120 Hz or higher in binocular mode. In one embodiment, an LED array of infrared lights are used as illumination resources for producing a corneal reflection.

The scene camera C2 in certain embodiments is similar to the cameras used in conventional head-mounted eye tracking systems, however, a depth sensor (e.g. depth sensor 11 in FIG. 1) is added to measure the depth of the objects in the scene, providing the distance between the objects and the camera. In one embodiment, hybrid information can be combined with RGB images and depth maps associated with each pixel on the RGB images to calculate the depth of objects within the scene. For example, depth sensors along with infrared laser projectors can be utilized to capture depth information independently from the visible light images. The depth information from sensors or infrared can be overlaid with the RGB images to associate certain objects with a particular depth. The sensing range of the depth sensor can be adjustable to fit different scenarios. In one embodiment, a device such as a normal webcam can be utilized to capture moving objects in the scene.

The scene may or may not be bounded (it is shown as bounded in FIG. 1 merely for illustrative purposes). For example, the scene could be one side of a room at a medical or research facility, an outdoor environment, such as a park or a city street, an underwater environment, a staged 3D environment, or an augmented virtual reality with virtual objects mixed with real physical objects. Although embodiments of the invention are an improvement over scenes that are projected to the subject via a screen, embodiments of the invention can include one or more screens within the 3D environment for observation by the subject. Further, the scene and/or the subject does not have to be static or stationary, since the scene camera C2 captures images of the scene in real time and is time synced with the eye camera C1. Advantageously, embodiments of the invention allow for dynamic scenes, such as a subject driving a car through a city street, or observation of a staged scene having moving objects.

In certain embodiments, the control unit 16 includes a processor, a memory unit, and communication ports, including the ports for operably connecting the cameras C1, C2 to the control unit 16. As would be understood by those having ordinary skill in the art, communication between components of the system can be implemented via wireless communication. The computer operable components of the eye tracking system may reside entirely on a single computing device, or may reside on a central server and run on any number of end-subject devices via a communications network that can include a cloud server. The computing devices may include all hardware and software typically found on computing devices for storing data and running programs, and for sending and receiving data over a network, if needed. The method of tracking gaze position disclosed herein can run through software accessible by or stored on the control unit. A system platform for performing and executing the methods and algorithms for eye tracking disclosed herein. As contemplated herein, any computing device as would be understood by those skilled in the art may be used with the system, including desktop or mobile devices, laptops, desktops, tablets, smartphones or other wireless digital/cellular phones, or other thin client devices as would be understood by those skilled in the art.

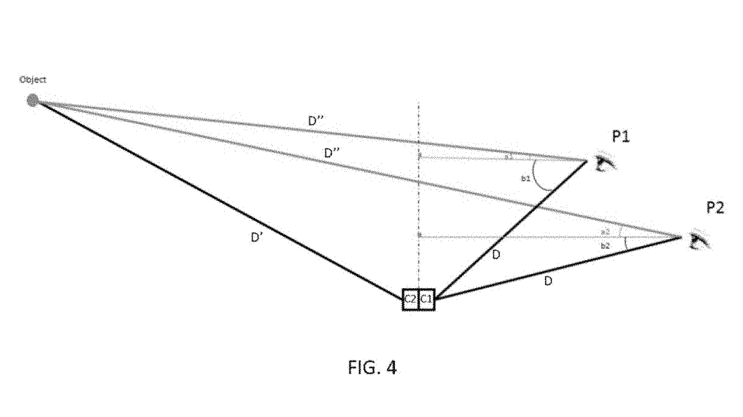

As stated above, in one embodiment, an infrared light can be used to illuminate the subject's eyes for corneal reflection, with reference now to FIGS. 3A-3C. One or more infrared illuminators 32 can be used (see FIG. 3A) so the when the eye camera C1 takes an image of the subject's eye 200, a corneal reflection 204 is produced in the pupil area 202 (see FIG. 3B). In one embodiment, there is a marker on the subject's forehead 210 such as a paper sticker with a pattern (in FIG. 3C shown as embedded squares, but could be any other type of marker or shape such as embedded triangles or concentric circles) used to determine the head angle of the subject. As in all non-head mounted eye tracking systems, it is necessary to compensate for the head movement of the subject. For the stability of calibration, it is necessary to have the information of the distance between the eye camera and the subject, as well as the head movement in 3D space (e.g. pitch, roll and yaw). Face detection algorithms known in the art can be applied to find the subject's face and eyes areas, and with existing feature detection methods, a triangle geometry structure can be found on the face for calculation of distance and 3D movement. For example, left eye, right eye and nose tip can form a triangle pattern. Alternatively, a geometry pattern can be defined with external markers (e.g. paper stickers as shown in FIG. 3B) to find the head distance and 3D head movement. As illustrated in FIG. 4, the distance between the eye camera C1 and the subject (shown by lines labeled D between C1 relative to subject's eyes shown in two alternate positions P1, P2) is provided by the pattern size detected on the subject's face. The subject's face to eye camera angles are also provided by the rotation angle of the pattern on face (b1, b2). This technique for determining gaze position is discussed in further detail below. Both single eye accommodation and techniques for accommodation using both eyes can be utilized.

In one embodiment, calibration is applied in order to map the subject's eye movement on the eye camera images to where the subject is fixating on the scene image. Before this calibration procedure, the software system of the eye tracker measures characteristics of shapes, light refraction and reflection properties of the different parts of the subject's eyes, and the information will be used to identify the corneal reflection and pupil location. During the calibration, the subject is asked to look at specific points in the three dimensional space of the visual scene, also known as calibration targets. These 3D calibration targets are located at different depths. The relative positions of the corneal reflection and pupil location are measured as the subject looks at calibration targets. This calibration finds correspondence where the corneal and pupil center are on the eye image and the known calibration targets on the scene image, when the subject is fixating, usually by a supervised fitting. During the rest of the experiment, the system interpolates between the calibrated landmarks to determine where the eye is fixating on the screen, then the tracker figures out where the eye must be looking, depending on the head movement and pupil position.

As shown in FIG. 4, the distance between the scene camera C2 and the object in the scene is provided by a depth sensor of the scene camera, the distance represented by D′. The distance (D) and angle (b1, b2) between eye camera C1 and subject's face is provided by the eye camera C1, while eye movement can be observed in eye images (a1, a2). Using the law of cosines, given two sides of a triangle and one of its angles, the length of the third side can be calculated, which is the distance between the object and the subject (D″). The association between the distance of the subject and scene object (D″) and the distance of left-right eye distance (binocular convergence) is constructed into the calibration model. With all the information compiled in the calibration model, even when the subject moves from position P1 to position P2 with changes of distance and angle, the validity of the calibration remains.

In one embodiment, depth and eye accommodation techniques are utilized when processing images of the subject. Eye accommodation happens when human subject changes visual focus distance. Mechanisms such as ciliary muscle regulate the change of lens shape. Changes in lens shape, pupil size and vergence during eye accommodation can be utilized in order to obtain a clear image on retina. When looking at a nearby object, the lens bends into large curvature and increases its reflective power; whereas when looking at a far object, the lens flattens into small curvature and decreases its reflective power. During eye accommodation, the pupil size changes like the aperture of a camera to control the periphery lights entering the eye. When looking at a nearby objects, the pupil constricts to a smaller size to reduce lights entering the periphery area of the eye, and by doing so, minimizes the interference of periphery lights to the center focusing (and vice versa for far distance object). Binocular convergence, which is the vergence of the relative viewing angle of the left and right eye, also helps to obtain stable image in our visual system. Any of these three changes or the combination of these changes can be used to measure the depth that the subject is subjectively viewing. Using binocular convergence as an example, any one or combination of the following can be used: the convergence of the left and right eye can be measured by the distance between the left and right pupil, the left and right iris in a 2 dimension image, the distance and angle between the left and right pupil, or the left and right iris in a 3 dimensional model.

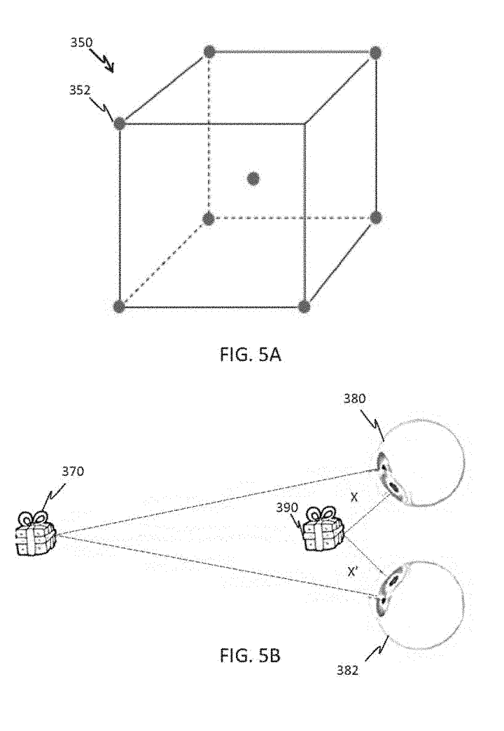

Based on the distance between the left and right pupil, the convergence of the subject's eyes can be observed, and the depth that the subject is observing can be calculated. For instance, with reference to FIGS. 5A and 5B, a scene 350 may have various objects (dots 352) setup at various different depth levels. The objects in the scene as viewed from the scene camera C2 can be integrated into the calibration model. When one object 370 is observed that is further back into the scene relative to a second object 390 positioned closer to the subject, the system will be able to determine which object the subject is focused on at any given time based on an observation of the relative convergence angles X, X′ via the eye camera C1. From the relevant position of both eyes, the system can interpret the convergence of the subject, and then integrate the depth of the object that the subject is observing into the calibration model. Advantageously, instead of 2D calibration on a surface such as a display screen, object targets are calibrated in a 3D space.

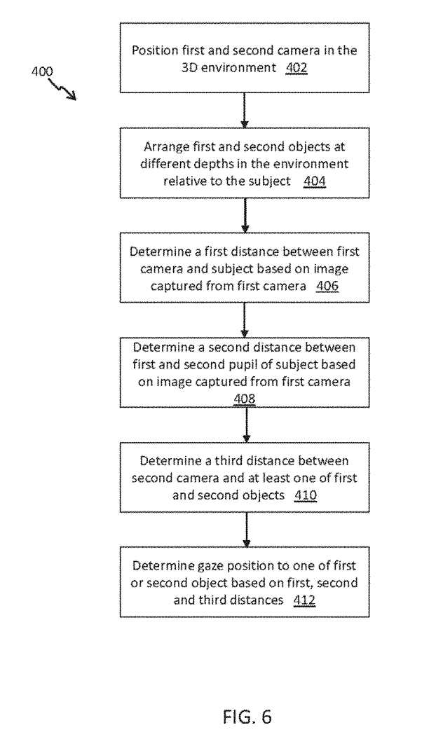

A method 400 for gaze tracking is shown in FIG. 6 according to one embodiment. The method includes positioning a first camera and a second camera in an environment including a subject, a first object and a second object 402. The first and second objects are arranged at different depths in the environment relative to the subject 404. A first distance is determined between the first camera and the subject based on an image captured from the first camera 406. A second distance is determined between a first pupil of the subject and second pupil of the subject based on an image captured from the first camera 408. A third distance is determined between the second camera and at least one of the first and second objects 410. Finally, a gaze position to one of the first and second object is determined based on the first, second and third distance 412. In one embodiment, the first camera and the second camera are positioned back to back. In one embodiment, the first camera and the second camera are spaced apart. In one embodiment, both the first and second cameras are fixed to a position that is disconnected from the subject. In one embodiment, the method an infrared light is positioned near at least one of the first and second object. In one embodiment, the first and second pupil are illuminated with infrared light. In one embodiment, a corneal reflection of the infrared light is detected. In one embodiment, movement of the subject is tracked using the first camera. In one embodiment, movement of at least one of the first and second object is tracked using the second camera. In one embodiment, the first and second cameras are mounted in a moving vehicle, and the two objects (e.g. a parked car and a pedestrian on the street) are outside of the moving vehicle. In one embodiment, head movement is tracked by utilizing a position marker affixed to the subject. In one embodiment, a distance between the first camera and the subject is determined based on a size of the position marker captured by the first camera. In one embodiment, head movement is tracked by utilizing a face detection and facial feature based algorithm.

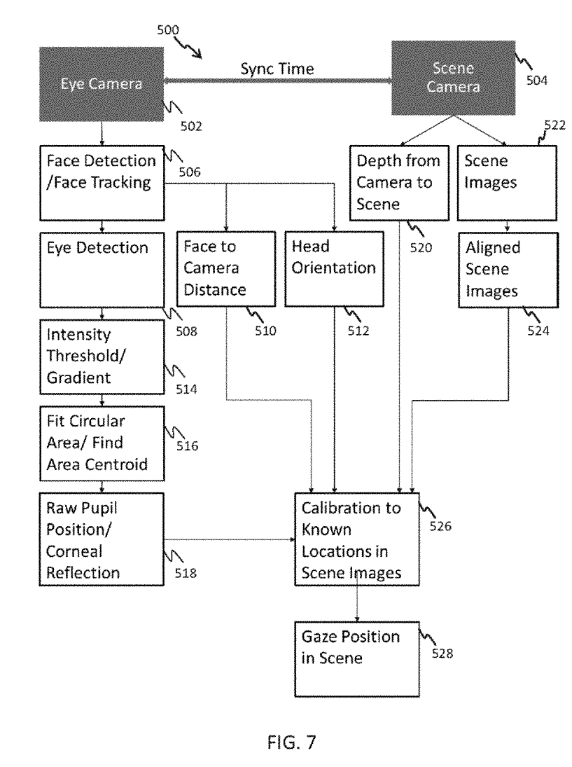

A method 500 for gaze tracking is shown in FIG. 7 according to one embodiment. The eye camera 502 and scene camera 504 are time synced with one another. Images from the eye camera 502 are used to first perform face detection and face tracking functions. Once the face is detected, a distance between the face and the camera is determined 510, and the head orientation is also determined 512. Eyes are detected 508, and an intensity threshold and/or gradient is determined 514 to calculate a fit and/or center 516 for finding raw pupil position and corneal reflection 518. With respect to images taken by the scene camera 504, depths from the camera to scene objects are determined 520. Scene images are also recorded 522 and aligned 524 relative to the predetermined calibration to known scene location and distance 526. The calibration also takes into account the observed head orientation 512, face to camera distance 510 and pupil position/corneal reflection 518 from the eye camera 502. Accordingly, the gaze position to an object in the scene can be determined 528. Embodiments of this method can be implemented using a software platform such as Matlab or any other programming language, for example, python, java or C/C#/C++ and so on. OpenCV is also a well-known Computer Vision toolbox and software platform that is helpful for face and eye region detection.

Embodiments of the system have many advantages over conventional systems. As conventional tracking systems are typically trying to access the first-person view with cameras positioned close to the subject's head, embodiments of the invention utilize cameras that can be positioned away from the subject at a third-person vantage point. Further, the systems described herein allow for a large range of flexibility in camera placement. Not only can the cameras be placed away from the subject, they can also be separated from each other. This allows for maximum flexibility and portability in observational environments. Further, in one embodiment, the system utilizes small cameras that can communicate remotely with a control unit, allowing for flexibility in mounting options for each camera. Further, regarding calibration, the calibration targets are not limited to a 2D surface, and instead, the calibration targets can be widespread throughout 3D locations in a real-life scene.

Embodiments of the systems and methods disclosed herein can be advantageously utilized in a number of applications. In certain instances, the embodiments are implemented while monitoring and recording a subject's movement in an interview or when the subject is watching real world activities. Interviews can be used for example for diagnosis or intervention of psychiatric or neuropsychological disorders (Autism, ADHD, Depression), or psychology or cognitive research. It's very helpful to provide the subject's or patient's eye movement in a real-life environment, especially in a no-invasive, no-contact, remote way. The eye movement data will facilitate more accurate diagnosis for the clinician, leading to the delivery of the best intervention. Proper invention will set the subject free from the anxiety and stress of wearing medical devices. Embodiments of the system allow recording of a subject's eye movement without their notice. Embodiments disclosed herein also provide real-time data and statistics to clinician or experimenter display devices, for example, laptops, tablets or google glass. Advantageously, the clinician or experimenter can have accurate detection and recording of the subject in real time, for example, number of eye contacts or looked away times.

Being easy to use and low cost, embodiments of the invention can be used not only in professional settings, but at home or in cars as well. It can be applied in monitoring and changing driving behavior. Conventional head mounted eye trackers have been applied in driving pattern analysis to find out where the driver is paying attention to in difference situations. However, due to the inconvenience and awareness of wearing the head mounted device, restricted head mounted eye tracker technology can only happen in simulation or research settings. Embodiments of the invention can advantageously utilize an eye camera installed on a position to observe the drivers eyes while the scene camera captures the driver's view of the road and surrounding environment. Eye movement monitoring and recording can happen in any car without wearing any device. In certain embodiments, the invention is implemented in a car to monitor and assist new drivers or teenagers by checking to see if they looked at important signs or events at the correct time. Further, systems and methods of the invention can be implemented for longer sessions (not the conventional 30 experimental session) over the course of months in the subjects own car, enabling the review and trajectory of changing of driving behavior.

In one embodiment, the systems and method are implemented in augmented reality (AR), which is a live direct or indirect view of a physical, real-world environment whose elements are augmented (or supplemented) by computer-generated sensory input such as sound, video, graphics or GPS data. In other words, augmented reality includes a blend of real world environments and object with computer simulated virtual objects. Applications for augmented reality include a wide range of settings that integrate information, experience and reality, such as for example retail shopping, education, travel, navigation, advertising and video gaming. Considering that computer-generated elements are assigned to certain locations by a computer that communicates with the control unit, this depth information is also known to the system, and the system can track the subject's eye movement in augmented reality among a mix of real-world 3D objects as well as virtual 3D objects in augmented reality. Embodiments of systems and method described herein for use with augmented reality environments can be used with or without a head mounted eye camera.

In one embodiment, the system utilizes a head mounted eye camera. The head mounted eye camera mode attaches the eye camera in the proximity of the subject's eyes, attached by a head gear or similar type of fitting known in the art. 3-dimensional real-world eye tracking for the head mounted eye camera mode can be performed using the methods disclosed herein.

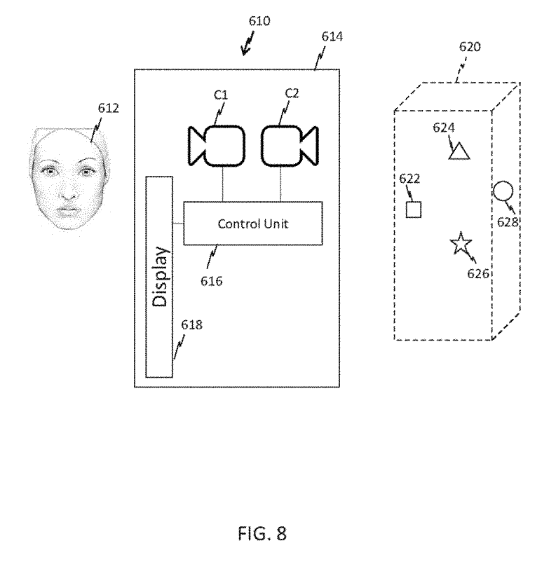

In one embodiment, as shown in FIG. 8, a handheld system 610 utilizes first C1 and second C2 cameras, which are first and second imaging units integrated into a handheld mobile device 614, such as a smartphone or a tablet. The first camera C1 is an eye camera trained on the subject 612, which in certain embodiments is integrated in the handheld device 614 on the same side as a display 618. The display 618 is electrically coupled to the control unit 616 along with the first and second cameras C1, C2. The second camera C2 is the scene camera trained on the 3D scene 620, which in certain embodiments is integrated in the handheld device 614 on the side opposite the first camera C1. In certain embodiments, the scene camera C2 displays its images on the display 618, which is facing the user 612. In certain embodiments, the mobile device 614 includes a depth sensor (not shown) for determining the depth of objects 622, 624, 626, 628 in the scene 620. Functionality of handheld device embodiments can be similar to the previous embodiments described above. In certain embodiments, a depth sensor is not utilized, and the scene camera C2 uses an estimation algorithm that relies on the objects' known sizes within the image to estimate object's distance. For example, if the actual size of the object is known, distance can be calculated according to its size on the scene image. Another example, if the object is moving, its relative distance can be determined using temporal parallax calculation. In certain embodiments, only relative distance is necessary and will be calculated.

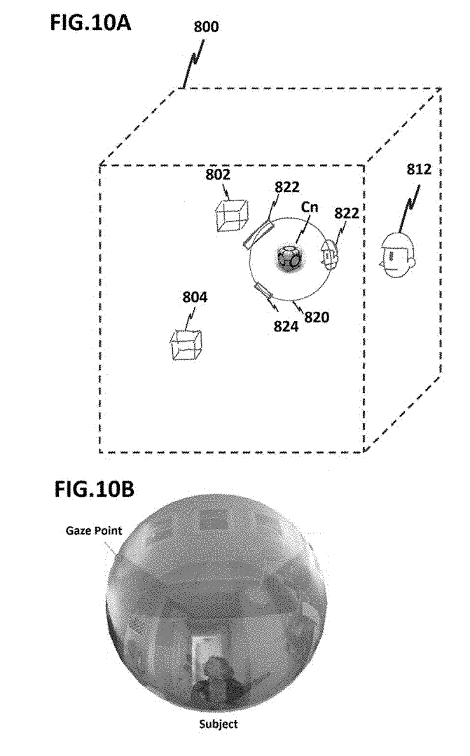

In one embodiment, as shown in FIG. 10a, first imaging unit and second imaging unit are integrated into on one physical device Cn, such as a 360 degree camera. The 360 degree camera has multiple imaging subsystems and forms a panorama display 820 of the 3D scene 800. Some of the imaging subsystems is trained on subject 812, while the some of the imaging subsystems are trained on the 3D objects 802, 804. In the 3D scene 800 all objects 802, 804 and the subject 812 form their correspondent projections 822, 824, 822 on the panorama display 820. In certain embodiments, a depth sensor is not utilized, and more than one imaging subsystems trained on the 3D objects uses 3D reconstruction algorithm to estimate object's distance. For example, the distance can be calculated from differences between the pictures from two imaging subsystems and their technical parameters of focal length and distance between the two imaging subsystems. In certain embodiments, these imaging subsystems composed of the same lens and imaging sensors, therefore sizes of imaging sensors and focal lengths are unified. The angular size of each object's projection varies on the panorama display, depending on the original size of the object and distance from the object to the camera. In certain embodiments, only relative distance is necessary and will be calculated.

As a personalized device, embodiments of the invention can be integrated into a gaze-controlled smart companion for disabled, paralyzed or locked in patients. This system according to embodiments described herein can provide an accurate 3D location of where the patient is looking and facilitate communication. For example, visitors could ask “Do you like Mary's new earrings?” if the patient is looking at them. Feedback of the patient can be provided by looking at different options on screen. With this system, patients with limited motor ability and language skill can give their instruction to caregivers or other smart devices, for example, by looking at the lights and curtain to choose to turn on lights, open curtains. In combination of automatic object recognition, this system can identify the objects and automatically recalibrate itself to improve accuracy through time.

### Experimental Examples

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

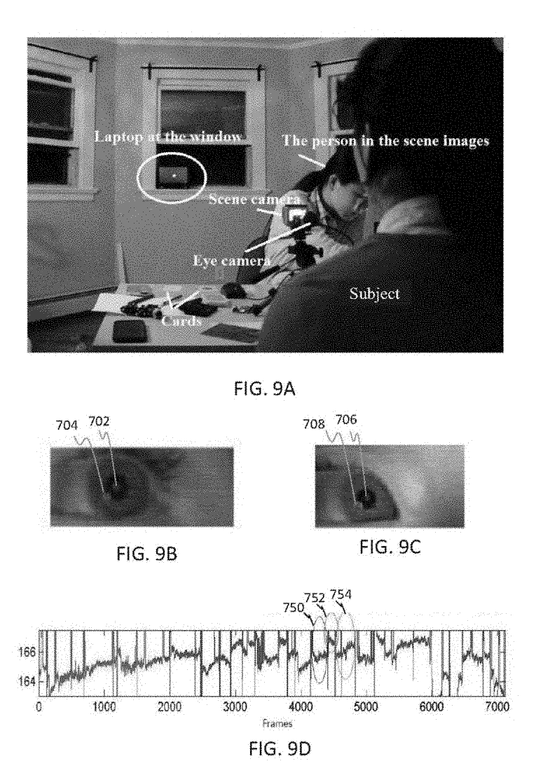

In the experimental setup shown in FIG. 9A, various objects including cards on a table and a laptop at the window were positioned at various depths in the 3D scene. An eye camera and a scene camera were placed back to back on a table between positioned in the room between the interviewer and the subject. As shown in left and right eye images of FIGS. 9B and 9C respectively, the corneal reflections 704, 708 are identified near the pupil areas 702, 706 by the eye camera. Measurements and calculations according to the methods described herein are conducted to determine where the subject is looking. The distance between the pupils while looking at the cards is represented by the section of the graph of FIG. 9D captured in the first circle 750. The distance between the pupils while looking at the laptop is represented by the section of the graph captured in the second circle 752. The third circle 752 represents the section of the graph when the subject first looked at the person conducting the interview, then moved their focus to the laptop in the back of the room.

In the experimental setup shown in FIG. 10B, a 360 camera is applied. In the real world scene all objects in a room and the subject form their correspondent projections on the panorama display, similar as in the illustrative figure FIG. 10A. All imaging subsystems have the same focal lengths and imaging sensors and the position differences of the imaging subsystems are fixed and known. Relative distances of the objects and subject is calculated based on the differences on pictures from different imaging subsystems. The casted ray from the subject represents the gaze direction of the subject, the intersection point of the casted ray and the panorama display of the scene is the gaze location on the real world scene of the subject.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A system for tracking gaze position of a subject comprising:
a first imaging unit, a second imaging unit operably connected to a control unit;
wherein the first imaging unit is configured to acquire subject image data, the second imaging unit is configured to acquire object image data, and to acquire object depth data; and
wherein the control unit is configured to receive the subject image data, the object image data and the object depth data and calculate a gaze position based on the received subject image data, object image data and object depth data.

2. The system of claim 1, wherein the first imaging units is an infrared imaging unit.

3. The system of claim 1, wherein the second image unit is composed of two or more imaging subsystems.

4. The system of claim 3, wherein the second image unit has a depth sensor that provides object depth data.

5. The system of claim 3, wherein the second image unit provides relative distances.

6. The system of claim 1, wherein at least one of the first and second imaging units include a wireless transmission component for wireless communication with the control unit.

7. The system of claim 1 further comprising:
an infrared light source for generating a corneal reflection that is captured by the first imaging unit.

8. A method for tracking gaze position of a subject comprising:
positioning a first imaging unit and a second imaging unit in an environment comprising a subject, a first object and a second object, the first and second object at different depths in the environment relative to the subject;
determining a first distance between the first imaging unit and the subject based on an image captured from the first imaging unit;
determining a second distance between a first pupil of the subject and second pupil of the subject based on an image captured from the first imaging unit;
determining a third distance between the second imaging unit and at least one of the first and second object; and
determining a gaze position to one of the first and second objects based on the first, second and third distance.

9. The method of claim 8, wherein the first imaging unit and the second imaging unit are positioned back to back.

10. The method of claim 8, wherein the first imaging unit and the second imaging unit are spaced apart.

11. The method of claim 8, wherein both the first and second imaging units are fixed to a position that is disconnected from the subject.

12. The method of claim 8 further comprising:
positioning an infrared light near at least one of the first and second object.

13. The method of claim 8 further comprising:
illuminating the first and second pupil with infrared light.

14. The method of claim 13 further comprising:
detecting a corneal reflection of the infrared light.

15. The method of claim 8 further comprising:
tracking movement of the subject using the first imaging unit.

16. The method of claim 8 further comprising:
tracking movement of at least one of the first and second object using the second imaging unit.

17. The method of claim 8, wherein the first and second imaging units are mounted in a moving vehicle, and wherein the first and second objects are outside of the moving vehicle.

18. The method of claim 8 further comprising:
tracking head movement by utilizing a position marker affixed to the subject.

19. The method of claim 18, wherein a distance between the first imaging unit and the subject is determined based on a size of the position marker captured by the first imaging unit.

20. The method of claim 8 further comprising;
tracking head movement by utilizing a face detection and facial feature detection algorithm.
